(19) United States
(12) Patent Application Publication
(10) Pub. No.: US 2017/0196191 A1
(43) Pub. Date: Jul. 13, 2017

(54) Sassie Cat

(71) Applicant: James Alfred Giles, St. Augustine, FL (US)

(72) Inventor: James Alfred Giles, St. Augustine, FL (US)

(21) Appl. No.: 14/994,465

(22) Filed: Jan. 13, 2016

(51) Int. Cl. A01K 1/01; A61L 9/14

(52) U.S. Cl. CPC A01K 1/0107; A61L 9/14; A61L 2209/111

(57) ABSTRACT

The present invention provides a cat litter box having opposed front wall and rear wall, left side wall and right side wall, a top and a bottom with respective adjacent edges contiguous to one another to define an enclosure. Front wall has an opening to allow cat to enter and exit the cat litter box. Front wall also includes a door near bottom for removably inserting litter tray containing litter inside the cat litter box. The litter tray is positioned at an elevated level at a rim present on the inner left side wall, inner right side wall and inner rear wall. The litter tray with used litter can be easily removed from the cat litter box to dispose used litter hygienically without the need to touch the used litter. Windows are provided on walls to provide ventilation. Deodorizing agent is automatically sprayed on entry and exit of cat.

### CROSS REFERENCE TO RELATED APPLICATIONS

Not applicable.

### FEDERALLY SPONSORED RESEARCH AND DEVELOPMENT

Not applicable.

### MICROFICHE APPENDIX

Not applicable.

### BACKGROUND OF THE INVENTION

(1) Field of Invention

The present invention generally relates to a cat litter box. More particularly, the present invention relates to “an easy to use the cat litter box which allows easily cleaning the used litter without need to touch the used litter, provides sufficient ventilation and efficient solution of smell”.

(2) Background of Invention

Cats are difficult to be house trained and needs to be provided with a device for collecting and disposing their liquid and solid waste. Various types of litter boxes are available for use with domestic cats. A typical litter box is rectangular box like container that is filled with litter material like sand, clay, zeolite, clumping litter material or biodegradable material, including paper. However, the litter in these litter boxes tends to be smelly and hence create a nuisance for the user. Some litter boxes have been developed which solves the problem of smell or which aim to allow improved litter system. For example, U.S. Pat. No. 7,594,480 B2 issued to Laura Sutis Cressy on 2009 Sep. 29 discusses a method of deodorizing an animal litter box where the litter box is lined with an absorbent containing liner and utilizing an ozone generator for disinfecting the litter box. Similarly, U.S. Pat. No. 7,856,945 B2 issued to Uni-Charm Corporation on 2010 Dec. 28, discloses an animal litter box with a movable hood to allow removing the hood to clean up the box. Similarly, U.S. patent application Ser. No. 13/372,281 discusses a self-drying litter box with tray superposition arrangement with one or more ventilation openings for allowing air circulation to dry the litter and waste materials deposited in the trays. However, these litter boxes are difficult to use besides being difficult to clean. Also, many of these litter boxes being complex are difficult to manufacture. Further, many of these litter boxes lack aesthetic appearance. Hence, it is desired to provide a cat litter box which is easy to use, easy to clean, easy to manufacture. Also, the cat litter box should provide an efficient way of controlling smell in the cat litter box. Further, the cat litter box should provide ventilation for the cat. Furthermore, it is highly desired that the user can dispose the used litter without need to touch the used litter. Also, it is desired that the cat litter box has aesthetic appearance.

It is an aspect of the invention to overcome or alleviate a problem of the prior art.

This permits the use of the present invention which enhance the prior art of the cat litter box for ease of use, ease of cleaning, easy manufacturing, efficient control of smell and sufficient ventilation for cat.

### BRIEF SUMMARY OF THE INVENTION

In view of the foregoing, one aspect of the various disclosed embodiments in the present invention is to provide a cat litter box which allows easily cleaning the used litter without need to touch the used litter, provides sufficient ventilation and efficient solution of smell.

Preferably the cat litter box addresses, or at least ameliorates one or more of the problems described above. To this end, the present invention related to a cat litter box, with some unique aspects is disclosed.

Accordingly, it is a primary object of the present invention to provide a cat litter box which is easy to use and easy to clean. The cat litter box provides a receptacle for removably keeping a litter tray inside. The cat is allowed to easily enter the cat litter box and use the litter stored in a litter tray positioned inside the cat litter box. The litter tray with used litter can be easily removed from the cat litter box and the used litter can be disposed hygienically without the need to touch the used litter.

It is another object of the present invention that the cat litter box has a deodorizer which automatically spray deodorizing agent on entry and exit of the cat.

It is yet another object of the present invention that the cat litter box is well ventilated.

It is yet another object of the present invention that the cat litter box is easy to transport and position at any location inside a home.

It is still another object of the present invention that the cat litter box is easy to manufacture. The cat litter box is preferably a one piece structure.

Other objects of the present invention will become apparent from time to time throughout the specification as hereinafter related.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

To further clarify various aspects of some example embodiments of the present invention, a more particular description of the invention will be rendered by reference to specific embodiments thereof which are illustrated in the appended drawing. It is appreciated that the drawing depicts only illustrated embodiments of the invention and are therefore not to be considered limiting of its scope. The invention will be described and explained with additional specificity and detail through the use of the accompanying drawing in which:

FIG. 1 shows a perspective view of the front wall of a cat litter box with the door in closed state;

FIG. 2 shows a perspective view of the front wall of a cat litter box with the door in the opened state;

FIG. 3 shows a perspective view of the bottom of a cat litter box.

### DETAIL DESCRIPTION OF THE INVENTION

The following detailed description of the present invention is enabling teaching of the invention and its best, currently known embodiment. Those skilled in the art can understand that many changes can be made in the embodiments described, while still obtaining beneficial results of the present invention. It will also be apparent that some of the desired benefits of the present invention can be obtained by selecting some of the features of the invention while not utilizing other features. Accordingly, those working in the relevant art will recognize that many adaptations and modification to the present invention can be made and may be desired in certain circumstances, and are part of the present invention. Thus, the following description is provided as illustrative of the principle of the present invention.

Embodiments of the present invention provide a cat litter box which allows easily cleaning the used litter without need to touch the used litter, provides sufficient ventilation and efficient solution of smell. Accordingly, the specific embodiments discussed herein are merely illustrative of specific manners in which to make and use this invention, and are not intended to represent an exhaustive list of all possible structure and processes of the present invention.

Embodiments of the cat litter box are shown in the figures, and discussed below. While the structure and processes have been described with a certain degree of particularity, it is to be noted that many modifications may be made in the details of the structure and processes without departing from the spirit and scope of this disclosure. It is understood that the structure and processes are not limited to the embodiments set forth herein for purposes of exemplification.

Aspects of the present inventive subject matter are described with reference to the figures.

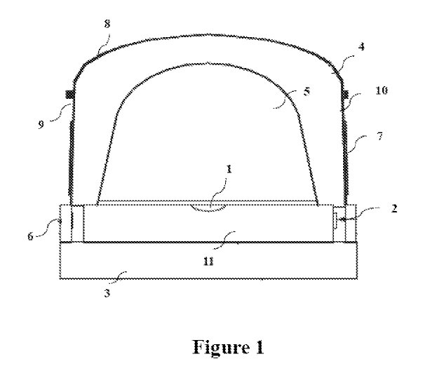

The present invention provides a cat litter box which provides a receptacle for removably keeping a litter tray inside. The cat is allowed to enter the cat litter box and use the litter stored in a litter tray positioned inside the cat litter box. Referring to FIG. 1 showing a preferred embodiment of the present invention, the cat litter box has opposed front wall (4) and rear wall (7), left side wall (9) and right side wall (10), a top (8) and a bottom with respective adjacent edges contiguous to one another to define an enclosure. The front wall (4) of the cat litter box has an opening (5) to allow the cat to enter the cat litter box to gain access to the litter stored in the litter tray (3) placed at the bottom of the cat litter box. The opening (5) is preferably arched to provide an aesthetic appearance. The opening may also be designed in other shapes. The opening (5) is preferably 8 inches high and 10 inches wide.

The cat litter box is provided with a sensor to detect the presence of the cat in the cat litter box. The cat litter box also has a deodorizer to spray to mask the litter smell when the cat enters or exit the cat litter box. The sensor is operationally connected to the deodorizer and once the sensor detects the entry of the cat in the cat litter box and exit of the cat from the cat litter box, the sensor sends a signal to the deodorizer to trigger release of the deodorizing agent in the form of spray. The deodorizer can be positioned preferably on the inner walls of the cat litter box. Alternatively a shelf can be provided on one of the inner walls of the cat litter box to allow positioning the deodorizer thereon.

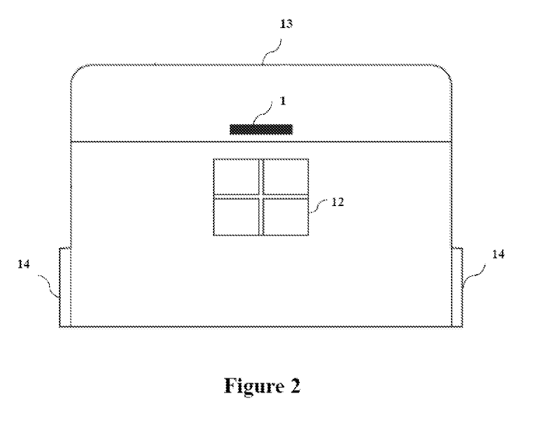

The cat litter box, preferably has an arched (13) top as shown in FIG. 2, the front side walls (5) of the cat litter box are preferably 18 inches high till the start of the arched top. The arched top of the cat litter box is preferably 20 inches long. The inner left side wall, inner right side wall and inner rear wall has a rim (16) near their bottom. When the litter tray (3) is inserted inside the cat litter box, the rim of the litter tray sits over the rim thereby positioning the litter tray at an elevated level.

The cat litter box is provided with a door at the front to allow insertion of the litter tray inside the cat litter box. The door (11) is preferably made of three members, including two side door members and one central door member (11). The two side door members are connected to the side walls, preferably with hinges, thereby closing and opening by swinging the side door members in and out respectively. The central door member is connected to the bottom of the cat litter box with hinges (2), thereby closing and opening by moving up and down respectively. The door has a handle to allow easily opening and closing the door for easy insertion and withdrawal of the litter tray. The handle is easy to be grasped. The handle (1) may preferably be attached to the top of the central door member (4). The side door member (6), is preferably 6 inches high and 1.5 inches wide. The central door member is preferably 6 inches high and 13 inches wide. In a preferred embodiment of the present invention, the door may be designed to be opened by first pulling up and then let down. The door, thus let down is then pushed into a slot in the front of the cat litter box.

A handle (1) is provided near the top end of the left side wall (4) and right side walls (14) to allow easy lifting and moving the cat litter box. Further, the cat litter box is provided with windows (12) to provide ventilation for the cat present inside the cat litter box and allowing the cat to look outside the cat litter box. Preferably, the cat litter box can have a window on the right side wall, the left side wall and the rear wall. Alternatively, the cat litter box can have multiple windows on the walls.

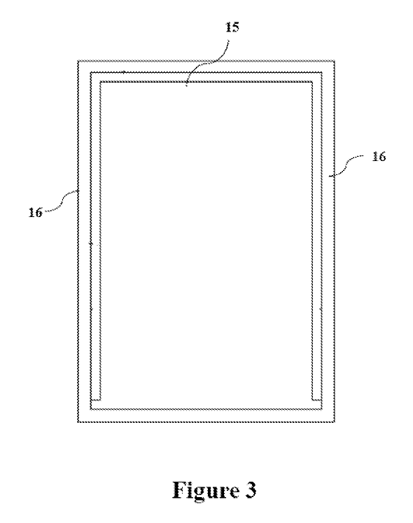

As shown in FIG. 3, the litter tray (3) used with the cat litter box is preferably made from plastic. The litter tray is sized preferably to be able to fit in a 13 gallon plastic bag with tie at the end to cover the tray. The cat litter box is very easy to use. Once the litter tray is covered with the plastic bag, the ties are looped (16) over the litter tray. Next, the litter is poured on the top of the plastic bag covering the litter receiving region of the litter tray. The window at the front of the cat litter box is opened. Then, the litter tray covered with the plastic bag and the having the litter filled up, is inserted in the cat litter box through the front of litter tray (3). The looped strings of the tie attached to the bag, fit in the quarter of an inch behind the door. The cat can easily steps over the door through the arched opening (15) into the box and use the litter stored inside. Once the cat has used the litter positioned inside the cat litter box, it is very easy to clean the used litter and throw away the used litter. To change the used litter, the door at the front of the cat litter box is opened. Then, the litter tray is pulled out through the opened door. The loop of the tie around the litter tray is removed to free the litter tray from the plastic bag. The litter tray is then pulled out of the plastic bag while leaving the used litter inside the plastic bag. The used litter thus captured inside the plastic bag can be tied up for disposal, say in garbage can.

The cat litter box of the present invention can be manufactured in different possible ways known in the art. Preferably, the cat litter box is one-piece structure. The walls and top of the cat litter box may further include designs, patterns including but not limited to images of cartoons, flowers, and other popular characters. The present invention thus provides an easy to use cat litter box which provides a comfortable, ventilated, and easy to access litter area for a cat. On the other hand, the cat litter box of the present invention allow easy handling by people for positioning at any location inside home, easy cleaning of the used litter in hygienic manner without having to touch the used litter, while deodorizing the interior of the cat litter box to control the smell.

It is to be understood that the above description is intended to be illustrative, and not restrictive. For example, the above discussed embodiments may be used in combination with each other. Many other embodiments will be apparent to those of skill in the art upon reviewing the above description.

Whereas, the construction and method have been described in relation to the figures of the drawings, it should be understood that other and further modifications, apart from those shown or suggested herein, may be made within the spirit and scope of this invention.

What is claimed is:

1. A cat litter box comprising:
opposed front wall and rear wall, left side wall and right side wall, a top and a bottom with respective adjacent edges contiguous to one another to define an enclosure;
the front wall including an opening to allow entry and exit of a cat;
the front wall including a door to allow removably insert a litter tray inside said cat litter box, said litter tray configured to receive and store litter; said door including a handle for opening and closing said door;
a rim near bottom of the inner left side wall, inner right side wall and inner rear wall to allow rim of said litter tray sit over said rim to position said litter tray at an elevated level;
at least one window in at least one of said left side wall, said right side wall, and said rear wall;
a sensor to detect the presence of the cat in said cat litter box;
a deodorizer to spray deodorizing agent, said deodorizer is operationally connected to sensor so that the deodorizer is triggered to release said deodorizing agent if said deodorizer receives a signal from said sensor;
wherein said left side wall including a handle near the top end, said right side wall including a second handle near the top end, said handles are configured to be gripped for lifting cat litter box.

2. The cat litter box of claim 1, wherein said door includes two side door member and one central door member, said side door members connected to said side walls of said cat litter box, said central door member connected to said bottom of said cat litter box.

3. The cat litter box of claim 1, wherein said door includes two side door member and one central door member, said side door members connected to said side walls of said cat litter box by hinges, said central door member connected to said bottom of said cat litter box by hinges.

4. The cat litter box of claim 1, wherein said door includes two side door member and one central door member, said side door member is 6 inches high and 1.5 inches wide, said central door member is 6 inches high and 13 inches wide.

5. The cat litter box of claim 1, wherein said cat litter box includes a slot in said front, said door is configured to be opened by first pulling up, then let down, and then pushed into said slot.

6. The cat litter box of claim 1, wherein said cat litter box includes a window in each of said left side wall, said right side wall, and said rear wall.

7. The cat litter box of claim 1, wherein said cat litter box includes multiple windows in at least one of said left side wall, said right side wall, and said rear wall.

8. The cat litter box of claim 1, wherein said litter tray is made from plastic.

9. The cat litter box of claim 1, wherein said litter tray is sized to be able to fit in a 13 gallon plastic bag, said litter tray configured to be covered by said plastic bag, said litter tray configured to receive litter on top of said plastic bag covering the litter receiving region of said litter tray.

10. The cat litter box of claim 1, wherein said litter tray is configured to be fit in a plastic bag with tie at the end of said plastic bag, said tie configured to be looped around said litter tray after said litter tray is inserted in said plastic bag, said litter tray configured to receive litter on top of said plastic bag covering the litter receiving region of said litter tray.

11. The cat litter box of claim 1, wherein said sensor is configured to detect entry and exit of cat and send signal to said deodorizer to trigger release of said deodorizing agent.

12. The cat litter box of claim 1, wherein said deodorizer is positioned on said inner walls of said cat litter box.

13. The cat litter box of claim 1, wherein said cat litter box includes a shelf one of said inner walls to allow positioning said deodorizer thereon.

14. The cat litter box of claim 1, wherein said opening is arched.

15. The cat litter box of claim 1, wherein said opening is 8 inches high and 10 inches wide.

16. The cat litter box of claim 1, wherein said top is arched.

17. The cat litter box of claim 1, wherein said top is arched, said side walls are 18 inches high till the start of the arched top, said arched top is 20 inches long.

18. The cat litter box of claim 1, wherein said cat litter box is one-piece structure.
